(19) United States
(12) Patent Application Publication
(10) Pub. No.: US 2004/0162456 A1
(43) Pub. Date: Aug. 19, 2004

(54) Method of preventing sedimentation of the crystals of gas hydrates

(76) Inventors: Patrick Gateau, Maurepas (FR); Anne Sinquin, Nanterre (FR); Virginie Beunat, Paris (FR); Regis Vilagines, Vienne (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/778,080

(22) Filed: Feb. 17, 2004

(30) Foreign Application Priority Data
Feb. 19, 2003 (FR) 03/02.053

(51) Int. Cl. G21F 9/00; C07C 9/00

(52) U.S. Cl. 588/15

(57) ABSTRACT

A process intended to prevent sedimentation of gas hydrates in petroleum production installations comprises adding to the petroleum effluent at least one additive making it possible to modify the rheological behaviour of the petroleum effluent to impart thereto a flow threshold fluid behaviour. The process uses polymer type organosoluble additives that can be:

di- or triblocks of which the blocks are non-compatible and of which one of the blocks is non-compatible with the solvent; or

microgels.

The invention concerns a process for preventing the sedimentation of crystals of gas hydrates in the low points and/or singular points of petroleum production installations by the use of at least one additive. The gases which form hydrates may in particular comprise for example methane, ethane, ethylene, propane, propene, n-butane, isobutane, H₂S and/or CO₂.

Those hydrates are formed when water occurs in the presence of the gas, either in the free state or in the state of being dissolved in a liquid phase such as a hydrocarbon and when the temperature of the mixture becomes lower than the thermodynamic temperature in respect of formation of the hydrates, which temperature is given for a known gas composition and a fixed pressure.

Formation of hydrates can be feared, in particular in the petroleum and gas industry where the conditions involving formation of the hydrates can occur jointly, in particular in production in deep water or in cold zones. Under those conditions hydrate crystals are observed to be formed, which very rapidly agglomerate and form blockages which stop hydrocarbon production.

Various solutions have been proposed to remedy those problems. In particular, when transporting liquid petroleum effluents in the presence of gas and water, the addition of dispersing additives is claimed (EP-B-0323774 and EP-B-0582507). Those products make it possible to transport the hydrate crystals formed in the form of a suspension while keeping them dispersed in the liquid hydrocarbon phase.

There are however conditions under which sedimentation of the particles is observed to occur. Those conditions are encountered when the hydrocarbon phase is both not very dense and not very viscous, as for example in the case of condensates, and when the flow speed is low, for example in a laminar condition, or indeed zero, for example in a situation when production is stopped. In those cases, sedimentation of the particles is observed at the low points and/or singular points in the conduit. Those particles, once sedimented, are difficult to put back into suspension, even when high flow speeds are then involved. Those sedimented particles give rise to a drop in production and, in the long run, by virtue of successive accumulation effects, they cause the conduit to become obstructed and production to be stopped.

The object of the present invention is to make it possible to avoid that sedimentation problem by modifying the rheological properties of the suspensions of crystals of hydrates by the addition of additives when that is necessary, that is to say in the presence of hydrates in a hydrocarbon phase which is of low viscosity and at hydrocarbon flow rates which are low or zero.

The additives used in the present invention are polymers which are soluble in the hydrocarbons. Those polymers are capable of forming weak intermolecular physical bonds. In the rest condition those bonds transform the hydrocarbon phase into a threshold fluid and under a low shearing effect those bonds impart a higher level of viscosity to the hydrocarbon phase. In those two cases sedimentation of the crystals of gas hydrates is avoided. In a flow condition, the intermolecular interactions are destroyed and the viscosity of the hydrocarbon phase falls, which permits the hydrocarbon to be caused to flow again and allows transport of the crystals of gas hydrates.

A definition of the threshold fluids can be found in the book: “An Introduction to Rheology”, H. A. Barnes, J. F. Hutton, K. Walters, Elsevier Science Publishers, 1989.

Those threshold fluid properties can be achieved for example by means of organosoluble block polymers in which the associated blocks exhibit a non-compatibility with each other and a non-compatibility of a block with the solvent. The following may be mentioned by way of example as a non-exhaustive list:

the diblock polymers hydrogenated styrene/butadiene, styrene/ethylene/propylene, or styrene/ethylene-butylene; or

the triblock polymers styrene/ethylene-butylene/styrene, styrene/butadiene/styrene or styrene/isoprene/styrene.

In all of those cases the rheological properties will be attained when the temperature of the medium is below the glass transition temperature (Tg) of the type of blocks making it possible to create weak intermolecular interactions.

Those same properties can also be achieved by means of microgels. Those microgels are polymers which are weakly cross-linked to form a solid particle and the periphery of which is composed of hanging chains of the same polymer, which are capable of association. The threshold fluid behaviour is achieved by entanglement of the hanging chains and the rheofluidifying character under a shearing effect corresponds to disentanglement of the chains. Those microgels may be for example polystyrenes or alkylpoly(meth)acrylates which are weakly cross-linked.

In both cases, when the shearing effect ceases, the block/block interactions in case 1 or entanglement of the chains in case 2 are reconstituted and the medium again exhibits a threshold.

The concentration of the additive is generally of 0.5 to 5% by mass with respect to the hydrocarbon phase of the petroleum effluent.

The following Examples illustrate the invention.

### EXAMPLE 1 (COMPARATIVE)

Using a 2 litre stainless steel reactor provided with two sapphire windows, an agitator, a temperature probe, a pressure regulator and an acquisition system capable of recording the pressure, gas consumption and temperature, 0.9 litre of a C6-C11 hydrocarbon cut containing 25.6% by mass of normal paraffins, 28.9% by mass of isoparaffins, 30.6% by mass of naphthenic compounds and 14.8% by mass of aromatic compounds is introduced. The procedure involves adding 0.9 gram of a dispersing additive as described in European patent EP 0 582 507, 0.1 litre of water and 20 grams of sand, the latter serving as an agent for nucleation of the methane hydrates. The agitation speed is regulated at 800 rpm and the reactor is put under a pressure of 70 bars of methane. The temperature of the reactor is lowered at a rate of 5° C./hour to 0° C., the pressure being maintained at 70 bars by the addition of methane. Under those conditions the hydrates are formed at 4.9° C.

When the temperature reaches 0° C. agitation is stopped and sedimentation of the hydrates is observed.

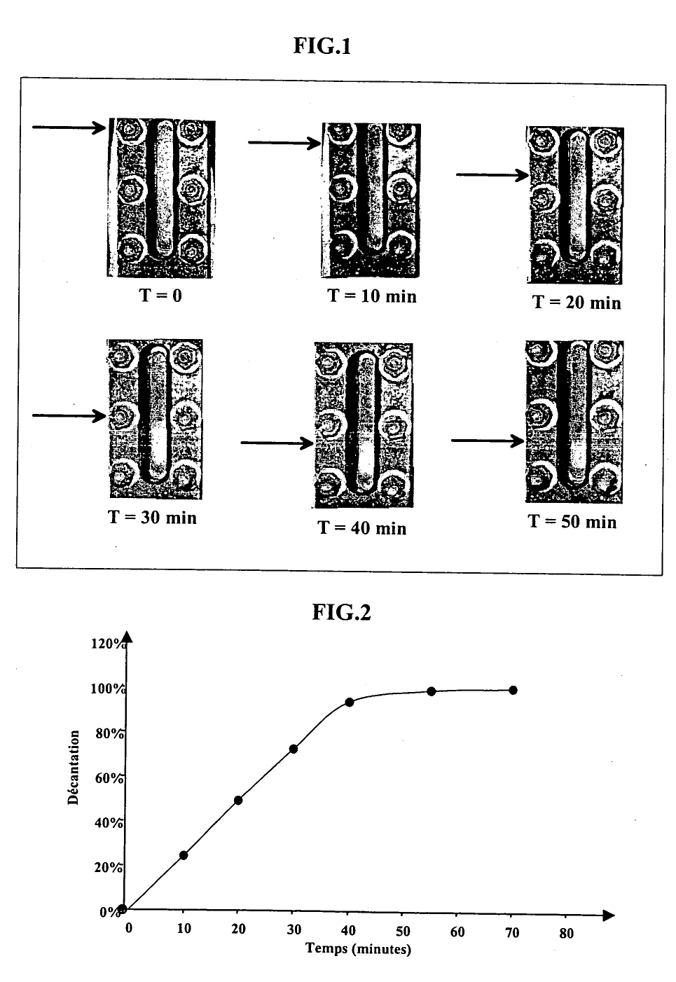

FIG. 1 shows the course of settlement of the hydrates. In FIG. 1 the arrows indicate the upper level of the crystals.

By relating [1-h/H]/hₘᵢₙ as a function of time (h=height of the layer of hydrates, H=height of the window and hₘᵢₙ=height of the sedimented layer of hydrates) it is observed that, under those conditions, the hydrates have experienced complete sedimentation in a period of 40 minutes (see FIG. 2).

### EXAMPLE 2

Example 1 is repeated with the addition to the hydrocarbon cut- of 3% by mass of a diblock copolymer styrene/ethylene-propylene containing 37% of styrene (Kraton® KG1701).

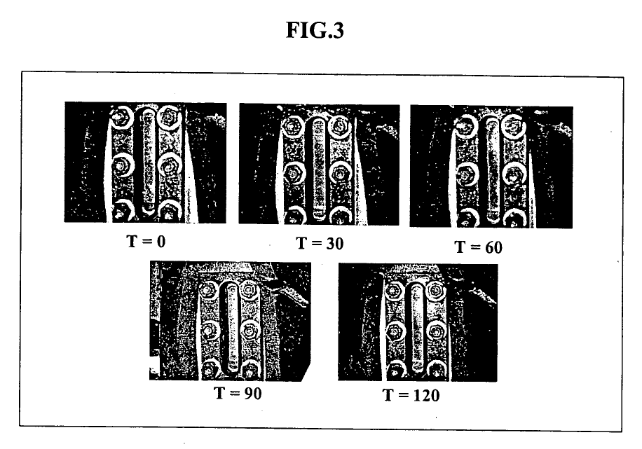

FIG. 3 shows the course of settlement of the hydrates. Under those conditions, no settlement of the crystals of hydrates is observed during a 2 hour period of stopping agitation.

1. A process for preventing the sedimentation of crystals of gas hydrates in the low points and/or singular points of petroleum production installations, characterised in that it comprises the addition to the petroleum effluent of at least one additive permitting modification of the rheological behaviour of the petroleum effluent to impart thereto a flow threshold fluid behaviour.

2. A process according to claim 1 characterised in that the additive is selected from polymers which are soluble in hydrocarbons.

3. A process according to claim 1 or claim 2 characterised in that the additive is selected from organosoluble block polymers in which the associated blocks exhibit a non-compatibility with each other and a non-compatibility of a block with the solvent.

4. A process according to claim 3 characterised in that the additive is a diblock polymer selected from hydrogenated styrene/butadiene polymers, styrene/ethylene-propylene polymers and styrene/ethylene-butylene polymers.

5. A process according to claim 3 characterised in that the additive is a triblock polymer selected from styrene/ethylene-butylene/styrene polymers, styrene/butadiene/styrene polymers and styrene/isoprene/styrene polymers.

6. A process according to claim 1 or claim 2 characterised in that the additive is selected from microgels consisting of polymers which are weakly cross-linked to form a solid particle and the periphery of which is composed of hanging chains of the same polymer, which are capable of association.

7. A process according to claim 6 characterised in that the microgel is selected from polystyrenes and alkylpoly(meth)acrylates which are weakly cross-linked.

8. A process according to anyone of claims 1 to 7 characterised in that the concentration of the additive is of 0.5 to 5% by mass with respect to the hydrocarbon phase of the petroleum effluent.
